(19) United States
(12) Patent Application Publication
(10) Pub. No.: US 2008/0009723 A1
(43) Pub. Date: Jan. 10, 2008

(54) STORAGE AND REVIEW OF ULTRASOUND IMAGES AND LOOPS ON HEMODYNAMIC AND ELECTROPHYSIOLOGY WORKSTATIONS

(76) Inventors: Richard W. Schefelker, Menomonee Falls, WI (US); Claudio P. Mejia, Wauwatosa, WI (US); Scott R. Kosloske, Slinger, WI (US); Aaron J. Hill, Burlington, WI (US); Gregory Rachvalsky, Haifa (IL)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/558,003

(22) Filed: Nov. 9, 2006

Related U.S. Application Data
(60) Provisional application No. 60/800,500, filed on May 15, 2006.

(51) Int. Cl. A61B 8/00

(52) U.S. Cl. 600/437

(57) ABSTRACT

A system and device for obtaining and reviewing patient data and ultrasound images from a patient such that the patient data and ultrasound images can be viewed simultaneously. The ultrasound images are obtained by an ultrasound system in real-time during a procedure carried out on the patient. The physiology workstation receives patient data as well as the ultrasound images and stores both the patient data and the ultrasound images within the physiological recording system. The ultrasound images and patient data are also communicated to a central data storage device where the ultrasound images and patient data are stored and can be accessed by a remotely located reviewing station. A display contained on the physiology workstation allows both the ultrasound images and the patient data to be viewed at the physiology workstation. Preferably, the patient data and the ultrasound images include common, time-based synchronization data obtained from a synchronization signal such that the ultrasound images and patient data can be time-synchronized during subsequent viewing after the completion of the procedure.

### CROSS-REFERENCE TO RELATED APPLICATION

The present invention is based on and claims priority to U.S. Provisional Patent Application Ser. No. 60/800,500, filed on May 15, 2006.

### FIELD OF THE INVENTION

The subject matter described herein generally relates to physiology and imaging workstations, and more particularly to integrating the various physiology and imaging features and functionalities such that the separate systems can operate together.

### BACKGROUND OF THE INVENTION

Today, physiology workstations are used in catheter labs, hemodynamic (HD) labs and electrophysiology (EP) labs to conduct various tests and procedures. Sometimes, the laboratory is organized into a procedure room, a control room and a remote monitoring room. Alternatively, there may not be a separate control or remote monitoring room. Instead, a sterile area where the patient lies is in the center of the room, and located in another area of the same room are the EP system and HP system, stimulator, etc. When available, the control and remote monitoring rooms are isolated from the sterile environment of the procedure room and are shielded from the x-rays generated in the procedure room by certain types of imaging equipment, such as fluoroscopy or CT imaging equipment. Presently, physiology workstations located in either the procedure, control or monitoring rooms are attached through cables to sensors, catheters, and instruments related only to the study. For example, conventional workstations are directly attached to surface ECG leads, intercardiac leads provided on a catheter, pressure sensors provided on a catheter and the like. The workstation is also directly attached to a stimulator that induces stimulus signals through a pacing tip on the catheter, such as to induce pacing to the heart.

Presently, the physiology workstation operates entirely separate and independent from imaging systems provided, such as an ultrasound system. The ultrasound system is a stand-alone system positioned in the procedure room proximate the patient and is controlled and operated by the physician or designated operator. The ultrasound system is attached to an ultrasound catheter or a surface probe that obtains ultrasound images. The ultrasound system is directly attached to a second set of surface ECG leads, separate and distinct from the surface ECG leads connected to the EP workstation. The ultrasound images are displayed on a dedicated ultrasound monitor positioned directly on the stand-alone ultrasound system in the procedure room. The ultrasound monitor in the procedure room is separate and distinct from the monitors in the control and remote monitoring rooms. The ultrasound system has a separate user interface dedicated and specific to ultrasound features and functionality. The ultrasound system also includes entirely independent and dedicated processing hardware and software, memory and the like. Thus, today, EP and HD studies are performed utilizing a stand-alone ultrasound system that is separate and distinct from the electrophysiology workstation.

Thus, when ultrasound equipment is used in the hemodynamic or electrophysiology lab, the stand-alone ultrasound unit causes the attending physician to have to correlate the results from the two pieces of equipment independently. In many hemodynamic and electrophysiology labs, the lab is configured in a network environment that allows physicians and nurses to view and manipulate case data on the main acquisition system as well as on secondary review stations. When the ultrasound equipment is used independently within the lab, the physician cannot use all of the available work stations to view the ultrasound data concurrently with the other procedural data being obtained from the patient, such as waveforms.

In many cases, it is desirable for the clinician to view both the ultrasound images and the heart waveforms simultaneously so that the clinician can view the current status of a patient. In currently available equipment, the display that is showing the patient waveform data for either the hemodynamic or electrophysiology equipment does not show the ultrasound images, while the ultrasound display does not include the hemodynamic measurements as may be desired by the clinician. Thus, the clinician must constantly shift his or her attention between the two separate displays to obtain a full understanding of the patient's situation.

One of the significant challenges of utilizing separate physiology workstations and ultrasound systems is the time-based synchronization of the information received from the two systems. For example, the meaningful interpretation of an ultrasound abnormality can be accomplished only in the context of other time-synchronized hemodynamic parameters such as blood pressure. Presently, the stored images from an ultrasound unit are time-stamped and stored either in the ultrasound unit or at a remote location (e.g. a remote server). The time data stored with the ultrasound images is useful in reviewing the stored images at a later time or from a remote location, such as by trained technicians or interpreting physicians. Likewise, the hemodynamic or electrophysiology measurements from the physiology workstations are also stored and include time-based information such that the stored data can also be reviewed at a later time or from a remote location by trained technicians. However, the stored images from the ultrasound unit and the physiology workstation are not currently integrated with each other, such that the time-based stored information may not be correlated or synchronized with the other systems.

### BRIEF DESCRIPTION OF THE INVENTION

An embodiment of the present invention is a system and method that integrates the functionality of an ultrasound system and a physiology workstation, such as a hemodynamic or an electrophysiological workstation, is provided. The integrated system may include a single display that shows time-synchronized ultrasound images and patient data for review by a physician/clinician.

In one preferred embodiment, the ultrasound system and physiology workstation include a communication link that allows the ultrasound system and the physiology workstation to be synchronized with each other. The synchronization of the ultrasound system and the physiology workstation allows the images from the ultrasound system and the patient data from the physiology workstation to be time-synchronized. The ultrasound images and the patient data can then be stored in a central data storage device and/or within the physiology workstation for later retrieving and viewing after the completion of the procedure.

Specifically, the ultrasound images are received by the physiology workstation and are stored within the physiology workstation. After the ultrasound images and patient data have been stored within the physiology workstation, the physiology workstation can be used to display both the ultrasound images and patient data simultaneously on a combined display or on multiple displays. The time-synchronization of the ultrasound images and the patient data allows for analysis of the two types of information simultaneously by a treating clinician/physician. After the patient data and the ultrasound images are stored within the physiology workstation, the ultrasound images and patient data can be relayed to a central data storage device and stored therein. Typically, the central data storage device forms part of a network that can be accessed by remote review stations, each of which can include a display having the ability to simultaneously show both the patient data and the ultrasound images. In such a case, the ultrasound images and the patient data include time-synchronization data derived from a common signal such that the ultrasound images and the patient data can be synchronized prior to display. The ultrasound images and the patient data will be displayed on an integrated display device at either the physiology workstation or the review station. However, use of multiple displays to show the time-synchronized information from the ultrasound system and the physiology workstation also allows the clinician to obtain a complete understanding of a patient's condition at a time either before or after the ultrasound images were recorded.

### BRIEF DESCRIPTION OF THE DRAWINGS

The drawings illustrate the best mode presently contemplated of carrying out the invention. In the drawings:

FIG. 1 is a schematic illustration of the integration between an ultrasound system and a physiology workstation;

FIG. 2 is an alternate embodiment illustrating the communication between the physiology workstations and a central data storage device such that the ultrasound images and patient data can be accessed by remote, review stations;

FIG. 3 is a schematic illustration of one embodiment of the communication between the ultrasound system and the physiology workstation to provide time synchronization between the patient data and ultrasound images; and

FIG. 4 is a screen shot of a representative display from either the physiology workstation or one of the remote review stations showing the simultaneous, time synchronized display of the ultrasound images and patient data.

### DETAILED DESCRIPTION OF THE INVENTION

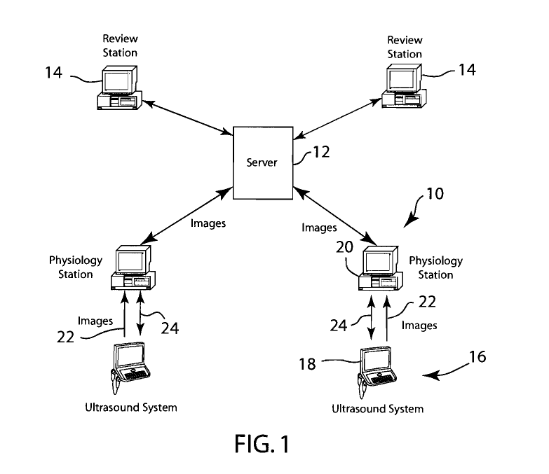

FIG. 1 illustrates a physiology workstation 10 that is typically located in the control room or procedural room of a catheter lab, hemodynamic (HD) lab or electrophysiology (EP) lab and is utilized in connection with HD, EP and ablation procedures, among other things. The physiology workstation 10 may integrate, among other things, real-time hemodynamic information, real-time intracardiac echography, fluoroscopic images, mapping data and pre-case planning CT and MR images. The physiology workstation 10 offers integrated collection and review of hemodynamic, EP, patient and mapping information as well as stored and real-time diagnostic images, ECG signals and IC signals.

The physiology workstation 10 is operable to acquire patient data, whether it be hemodynamic or electrophysiological data from the patient, such as during the performance of a catheterization procedure. In a networked system, the physiology workstation 10 communicates the patient data to a central data storage device or server 12 that stores the patient data for access and review by a physician from a remote review station 14. In such prior physiology workstations, the review station 14 allows a physician to review the patient data from a location remote to the physiology workstation 10 and analyze the data as required.

As an example, the physiology workstation 10 can receive signals from various different devices, such as intracardiac signals from EP catheters, patient signals, (e.g. from a blood pressure cuff, SPO2 monitor, temperature monitor, CO2 levels and the like), ECG signals from surface ECG leads, pressure signals from catheters and intracardiac signals. Further, the physiology workstation 10 can also receive fluoroscopic imaging data from a fluoroscopic system and ablation data from an ablation source and controller.

Due to the rapid advancements of ultrasound systems, procedures carried out in a catheter or EP lab often incorporate an ultrasound system 16 that provides the treating physician with additional information regarding the procedure being carried out in the catheter or EP lab. The ultrasound system 16 obtains ultrasound images from the patient, which are shown on a display screen 18 incorporated within the ultrasound system 16. During a procedure within the catheter or EP lab, the ultrasound images shown on the display 18 could only be compared with the patient data shown on the display screen 20 of the physiology workstation 10 by the physician either placing the two screens near each other and shifting focus between the two different data set displayed on the displays 18, 20. Once the procedure was complete, re-synchronization of the stored ultrasound images and the patient data was impossible.

In accordance with one embodiment, the ultrasound system 16 is in communication with the physiology workstation 10 such that ultrasound images from the ultrasound system 16 are received at the physiology workstation 10, as shown by the image transfer line 22.

Upon receiving the ultrasound images from the ultrasound system 16, the physiology workstation 10 initially stores the ultrasound images within a storage location within the physiology workstation 10. Specifically, each time an ultrasound image or loop is saved on the ultrasound system 16, the ultrasound image will automatically be sent to the physiology workstation 10 and stored therein.

As illustrated in FIG. 1, a time-base synchronization signal 24 is provided between the ultrasound system 16 and the physiology workstation 10 to synchronize the internal clocks of each system. The synchronization signal 24 allows the ultrasound images and the patient data from each of the systems 10, 16 to be stored with the same time-based synchronization data either within the physiology workstation 10 or at a separate, remote location. The synchronization of the ultrasound images from the ultrasound system 16 and the patient data from the physiology workstation 10 allows the two sets of information to be retrieved and displayed at a later time such that the information is time-synchronized and can be reviewed by a clinician as such. The ability of a clinician to review the ultrasound images and the patient data in synchronization allows the clinician to be presented with a complete picture of the procedure, which otherwise would have been unavailable.

In the embodiment shown in FIG. 1, the ultrasound images created by the ultrasound system 16 and the patient data from the physiology workstation 10 are synchronized with each other and stored within the physiology workstation 10 for later viewing. In addition to storing both the patient data and the ultrasound images within the physiology workstation 10, the physiology workstation 10 communicates the ultrasound images and the patient data to the central data storage device 12, which is part of the networked system. The central data storage device 12 can then be accessed by one of several review stations 14 such that a clinician located remotely from the physiology workstation can review the ultrasound images and patient data in synchronization to provide a better overall summary of the patient condition.

In the embodiment shown in FIG. 1, the central data storage device 12 can be any type of storage media. The remote review stations 14 can be in communication with the central data storage device 12 to retrieve and display the images from the ultrasound system 16 and the physiology workstation 10. The patient data and ultrasound images can be simultaneously displayed on the review station 14 for analysis by the clinician.

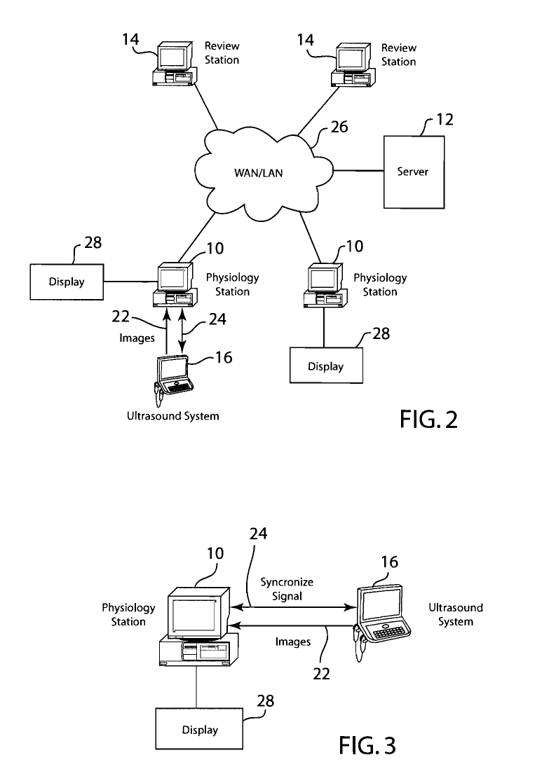

Referring now to FIG. 2, thereshown is yet another embodiment in which the ultrasound system 16 communicates ultrasound images 22 to the physiology workstation 10 and receives a synchronization signal 24. In the embodiment shown in FIG. 2, the physiology workstations 10 communicate to the central data storage device 12 through a network 26, such as a WAN or LAN interface. Each of the remote review stations 14 also communicates to the central data storage device 12 through the network 26. Since the physiology workstation 10 uploads both the patient data and the ultrasound images to the central data storage device 12, a clinician at the remote review stations 14 can view both the patient data and the ultrasound images that were obtained using the ultrasound system 16. Further, since both the patient data and the ultrasound images are stored with the time-based synchronization data, a reviewer at any one of the review stations 14 can review the ultrasound images and patient data in time-synchronization with each other. In this manner, the clinician is able to view the entire situation that occurred at the patient during the completed procedure.

As can be understood in the embodiments of FIGS. 1 and 2, the ultrasound system 16 and the physiology workstation 10 include a communication interface that allow the two systems 10, 16 to communicate with each other. Typically, when either of the systems 10, 16 is used with a patient, patient demographic information needs to be entered into the workstation or uploaded from the hospital information system (HIS). Since the ultrasound system 16 and physiology workstation 10 are in communication with each other, patient demographic data can be shared between the ultrasound system 16 and the physiology workstation 10. The shared patient demographic information eliminates the requirement that the clinician/physician enter duplicate information into each of the two systems 10, 16. Additionally, the communication between the two systems 10, 16 requires that only one of the ultrasound systems 16 and the physiology workstation 10 be connected to the hospital information system.

Referring now to FIG. 3, in a catheter or EP lab that is not part of a connected network, the physiology workstation 10, such as a hemodynamic or electrophysical workstation, acquires patient data from the patient during the performance of the procedure. The data obtained from the patient is typically shown on a display 28 for review by the treating clinician/physician. In accordance with the embodiment of the invention shown, the ultrasound system 16 is in communication with the physiology workstation 10 such that images from the ultrasound system 16 can be received and stored at the physiology workstation 10. The display 28 can take the form of many different embodiments, such as a single display or a panel of displays connected to the same physiology workstation 10. In either case, since the physiology workstation 10 receives and stores the ultrasound images, both the patient data and the ultrasound images can be shown on the display 28. Preferably, the ultrasound images and the patient data can be displayed simultaneously on the display 28 and are time-synchronized based upon the synchronization signal 24 that is communicated between the ultrasound system 16 and the physiology workstation 10.

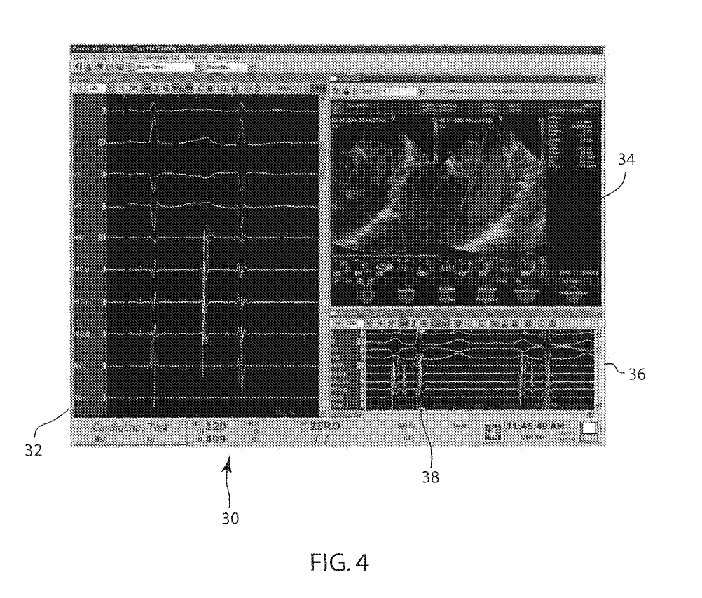

As described above, both the review station 14 and the physiology workstation 10 include a display that allows for the display of both the ultrasound images and the patient data. FIG. 4 illustrates the typical screen shot from the display screen 20 of the physiology workstation. The display screen 30 includes a patient physiology data display section 32 and an ultrasound display section 34. In the embodiment shown, the physiology data display section 32 illustrates various leads from an ECG monitor, while the ultrasound display section 34 illustrates ultrasound images taken during the performance of a procedure. As discussed previously, the synchronization signal used when recording both the patient data and the ultrasound images allows for the physiology data display section 32 and the ultrasound display section 34 to show time-synchronized data in each of the two separate sections 32, 34. In the embodiment shown in FIG. 4, the display screen 30 also includes an expanded timeline section 36 that shows a greater range of the hemodynamic patient data, part of which is magnified and shown in the physiology data display section 32. The timeline section 36 allows the clinician to view a greater range of patient data, while the indicator tabs 38 illustrate the current time being viewed in both of the physiology data display section 32 and the ultrasound display section 34.

As discussed previously, the combined display screen 30 that illustrates both the patient data and the ultrasound images can be shown on both the review station 14 and/or the physiology workstation 10. Thus, a clinician present at either the physiology workstation 10 or the review station 14 can review the ultrasound images and patient data in a time-synchronized manner to conduct an analysis of the patient being monitored.

As shown in FIG. 4, the physiology data display section 32 and the ultrasound display section 34 are shown as being incorporated into a single screen. However, it should be understood that the physiology workstation could include multiple monitors and the physiology data display section 32 and the ultrasound display section 34 could be displayed on separate monitors connected to the same physiology workstation. Alternatively, the physiology workstation 10 could be connected to a large display panel such as used in an operating room environment such that the hemodynamic or EP measurements and the ultrasound images could be simultaneously displayed and synchronized together for analysis by a treating physician. The use of the time-synchronization signal between the physiology workstation and the ultrasound system allows the patient data and ultrasound images to be time-correlated with each other for synchronous display at a later time. The time synchronization of the two different measurements allows a clinician to analyze the different types of measurements and come to a diagnosis/result based upon the combined information.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A system for obtaining and reviewing patient data and images from a patient, the system comprising:
at least one physiology workstation operable to obtain data from the patient;
an ultrasound system operable to obtain ultrasound data from the patient, the ultrasound system being in communication with the physiology workstation such that the ultrasound data is received by the physiology workstation and stored in the physiology workstation; and
wherein the physiology workstation includes a display operable to simultaneously display both the patient data and the ultrasound data from the patient.

2. The system of claim 1 wherein the physiology workstation is a hemodynamic workstation operable to obtain hemodynamic measurements from the patient.

3. The system of claim 1 wherein the physiology workstation is an electrophysiology workstation operable to obtain electrophysiology measurements from the patient.

4. The system of claim 1 wherein the patient data and the ultrasound data include time-based synchronization data, wherein the ultrasound data and the patient data can be simultaneously displayed on the integrated display of the physiology workstation based on the synchronization data.

5. The system of claim 1 further comprising:
a central data storage device in data communication with the physiology workstation to receive and store the patient data from the physiology workstation for the patient, wherein both the patient data and the ultrasound data are stored within the central data storage device; and
at least one review station in communication with the central data storage device to access and view the ultrasound data and the patient data for the patient.

6. The system of claim 5 wherein the review station includes an integrated display operable to simultaneously display both the patient data and the ultrasound data.

7. The system of claim 4 wherein the time-based synchronization data is the same for both the ultrasound data and the patient data.

8. The system of claim 7 wherein the synchronization data is recorded simultaneously with the ultrasound data and the patient data.

9. A method of obtaining ultrasound images from an ultrasound system and patient data from a physiology workstation, the method comprising the steps of:
operating the physiology workstation to obtain patient data from the patient;
operating the ultrasound system to obtain ultrasound images from the patient and communicating the ultrasound images to the physiology workstation;
storing the ultrasound images and the patient data in the physiology workstation; and
simultaneously displaying the patient data and the ultrasound data on a display of the physiology workstation.

10. The method of claim 9 further comprising the steps of:
providing a time-based synchronization signal between the ultrasound system and the physiology workstation;
correlating the ultrasound images and the patient data obtained by the ultrasound system and the physiology workstation to the synchronization signal; and
storing the ultrasound images and the patient data with the time-based synchronization signal in the physiology workstation.

11. The method of claim 10 further comprising the step of simultaneously displaying the ultrasound images and the patient data on the physiology workstation, wherein the ultrasound images and patient data are time-synchronized based on the synchronization signal.

12. The method of claim 11 wherein the physiology workstation is a hemodynamic monitoring system.

13. The method of claim 11 wherein the physiology workstation is an electrophysiology monitoring system.

14. The method of claim 9 further comprising the steps of:
communicating both the patient data and the ultrasound images from the physiology workstation to a central data storage device; and
storing the patient data and the ultrasound data in the central data storage device.

15. The method of claim 14 further comprising the steps of:
providing a time-based synchronization signal between the ultrasound system and the physiology workstation;
correlating the ultrasound images and the patient data obtained by the ultrasound system and the physiology workstation to the synchronization signal; and
storing the ultrasound images and the patient data with the time-based synchronization signal in the central data storage device.

16. The method of claim 10 further comprising the steps of:
accessing the central data storage device from a review station; and
displaying both the patient data and the ultrasound images at the receiving station.

17. The method of claim 16 further comprising the step of simultaneously displaying the ultrasound images and the patient data on the review station, wherein the ultrasound images and the patient data are time-synchronized based upon the synchronization signal.

18. A physiology system for medically recording patient data, comprising:
a physiology workstation operable to obtain patient data from the patient;
an ultrasound system operable to obtain an ultrasound image of a region of interest of the patient, wherein the ultrasound system is coupled to the physiology workstation to communicate the ultrasound image to the physiology workstation;
a display device integrated with the physiology workstation for displaying the physiology workstation and the ultrasound image; and
a data storage device integrated with the physiology workstation for storing the patient data and the ultrasound image;
wherein the patient data and the ultrasound image are obtained in connection with a common synchronization signal such that the ultrasound image and the patient data are time correlated.

19. The physiology system of claim 18 further comprising a central data storage device in communication with the physiology workstation to receive and store the patient data and the ultrasound images from the physiology workstation.

20. The physiology system of claim 17 further comprising at least one review station located remotely from the physiology workstation and in communication with the central data storage device, wherein the review station can obtain and simultaneously display the patient data and ultrasound image stored in the central data storage device.
